(19) United States
(12) Patent Application Publication
(10) Pub. No.: US 2009/0105762 A1
(43) Pub. Date: Apr. 23, 2009

(54) Dynamic stabilization member with fin supported segment

(76) Inventor: Roger P. Jackson, Prairie Village, KS (US)

Correspondence Address: LAW OFFICE OF JOHN C. MCMAHON, P.O. BOX 30069, KANSAS CITY, MO, 64112, US

(21) Appl. No.: 12/287,035

(22) Filed: Oct. 3, 2008

Related U.S. Application Data
(60) Provisional application No. 60/999,965, filed on Oct. 23, 2007.

(51) Int. Cl. A61B 17/70; A61F 2/44

(52) U.S. Cl. 606/246; 623/17.11

(57) ABSTRACT

A dynamic fixation medical implant having at least two bone anchors includes a dynamic longitudinal connecting member assembly having the following features: a pair of elongate segments, each segment having an abutment plate and a plurality of integral fins axially extending therefrom; and a molded elastomer that substantially surrounds the fins.

### CROSS-REFERENCE TO RELATED APPLICATIONS

This application claims the benefit of U.S. Provisional Patent Application Ser. No. 60/999,965, filed Oct. 23, 2007 and incorporated by reference herein.

### BACKGROUND OF THE INVENTION

The present invention is directed to dynamic fixation assemblies for use in bone surgery, particularly spinal surgery, and in particular to longitudinal connecting members and cooperating bone anchors or fasteners for such assemblies, the connecting members being attached to at least two bone anchors.

Historically, it has been common to fuse adjacent vertebrae that are placed in fixed relation by the installation therealong of bone screws or other bone anchors and cooperating longitudinal connecting members or other elongate members. Fusion results in the permanent immobilization of one or more of the intervertebral joints. Because the anchoring of bone screws, hooks and other types of anchors directly to a vertebra can result in significant forces being placed on the vertebra, and such forces may ultimately result in the loosening of the bone screw or other anchor from the vertebra, fusion allows for the growth and development of a bone counterpart to the longitudinal connecting member that can maintain the spine in the desired position even if the implants ultimately fail or are removed. Because fusion has been a desired component of spinal stabilization procedures, longitudinal connecting members have been designed that are of a material, size and shape to largely resist flexure, extension, torsion, distraction and compression, and thus substantially immobilize the portion of the spine that is to be fused. Thus, longitudinal connecting members are typically uniform along an entire length thereof, and usually made from a single or integral piece of material having a uniform diameter or width of a size to provide substantially rigid support in all planes.

Fusion, however, has some undesirable side effects. One apparent side effect is the immobilization of a portion of the spine. Furthermore, although fusion may result in a strengthened portion of the spine, it also has been linked to more rapid degeneration and even hyper-mobility and collapse of spinal motion segments that are adjacent to the portion of the spine being fused, reducing or eliminating the ability of such spinal joints to move in a more normal relation to one another. In certain instances, fusion has also failed to provide pain relief.

An alternative to fusion and the use of more rigid longitudinal connecting members or other rigid structure has been a “soft” or “dynamic” stabilization approach in which a flexible loop-, S-, C- or U-shaped member or a coil-like and/or a spring-like member is utilized as an elastic longitudinal connecting member fixed between a pair of pedicle screws in an attempt to create, as much as possible, a normal loading pattern between the vertebrae in flexion, extension, distraction, compression, side bending and torsion. Problems may arise with such devices, however, including tissue scarring, lack of adequate spinal support or being undesirably large or bulky when sized to provide adequate support, and lack of fatigue strength or endurance limit. Fatigue strength has been defined as the repeated loading and unloading of a specific stress on a material structure until it fails. Fatigue strength can be tensile or distraction, compression, shear, torsion, bending, or a combination of these.

Another type of soft or dynamic system known in the art includes bone anchors connected by flexible cords or strands, typically made from a plastic material. Such a cord or strand may be threaded through cannulated spacers that are disposed between adjacent bone anchors when such a cord or strand is implanted, tensioned and attached to the bone anchors. The spacers typically span the distance between bone anchors, providing limits on the bending movement of the cord or strand and thus strengthening and supporting the overall system. Such cord or strand-type systems require specialized bone anchors and tooling for tensioning and holding the cord or strand in the bone anchors. Although flexible, the cords or strands utilized in such systems do not allow for elastic distraction of the system once implanted because the cord or strand must be stretched or pulled to maximum tension in order to provide a stable, supportive system. Also, as currently designed, these systems do not provide any significant torsional resistance.

The complex dynamic conditions associated with spinal movement therefore provide quite a challenge for the design of elongate elastic longitudinal connecting members that exhibit an adequate fatigue strength to provide stabilization and protected motion of the spine, without fusion, and allow for some natural movement of the portion of the spine being reinforced and supported by the elongate elastic or flexible connecting member. A further challenge are situations in which a portion or length of the spine requires a more rigid stabilization, possibly including fusion, while another portion or length may be better supported by a more dynamic system that allows for protective movement.

### SUMMARY OF THE INVENTION

Longitudinal connecting member assemblies according to the invention for use between at least two bone anchors provide dynamic, protected motion of the spine and may be extended to provide additional dynamic sections or more rigid support along an adjacent length of the spine, with fusion, if desired. A longitudinal connecting member assembly according to the invention has a pair of elongate segments, each segment having at least one and up to a plurality of integral fins extending axially from an end of the segment. The fin or fins of each segment are oriented partially overlapping the fin or fins of the other elongate segment, the fins being spaced from one another. An elastic over-molded outer spacer is disposed about the fins of both segments and holds the segments together in spaced relation. One of the illustrated embodiments further includes an inner floating pin.

### OBJECTS AND ADVANTAGES OF THE INVENTION

Therefore, it is an object of the present invention to overcome one or more of the problems with bone attachment assemblies described above. An object of the invention is to provide dynamic medical implant stabilization assemblies having longitudinal connecting members that include a flexible portion that allows for bending, torsion, compression and distraction of the assembly. A further object of the invention is to provide dynamic medical implant longitudinal connecting members that may be utilized with a variety of bone screws, hooks and other bone anchors. Another object of the invention is to provide a more rigid or solid connecting member portion or segment, if desired, such as a solid rod portion integral to the flexible portion. Additionally, it is an object of the invention to provide a lightweight, reduced volume, low profile assembly including at least two bone anchors and a longitudinal connecting member therebetween. Furthermore, it is an object of the invention to provide apparatus and methods that are easy to use and especially adapted for the intended use thereof and wherein the apparatus are comparatively inexpensive to make and suitable for use.

Other objects and advantages of this invention will become apparent from the following description taken in conjunction with the accompanying drawings wherein are set forth, by way of illustration and example, certain embodiments of this invention.

The drawings constitute a part of this specification and include exemplary embodiments of the present invention and illustrate various objects and features thereof.

### BRIEF DESCRIPTION OF THE DRAWINGS

FIG. 1 is an enlarged and partial, exploded perspective view of a dynamic fixation connecting member assembly according to the invention including first and second elongate members, each with a finned end plate, an elongate core member and an outer molded spacer (not shown).

FIG. 2 is an enlarged front elevational view of one of the finned elongate members of FIG. 1.

FIG. 3 is an enlarged side elevational view of the elongate member of FIG. 2.

FIG. 4 is an enlarged opposite side elevational view of the elongate member of FIGS. 2 and 3.

FIG. 5 is an enlarged perspective view of the assembly of FIG. 1 shown in an assembled orientation prior to molding of the spacer therein.

FIG. 6 is an enlarged front elevational view of the assembly of FIG. 5

FIG. 7 is an enlarged front elevational view, similar to FIG. 6, with portions broken away to show the detail thereof.

FIG. 8 is an enlarged perspective view of the assembly of FIG. 1.

FIG. 9 is a reduced and partially exploded perspective view of the assembly of FIG. 7 shown with a pair of bone screws and cooperating closure structures.

FIG. 10 is an enlarged and partial perspective view of an alternative dynamic fixation connecting member assembly according to the invention including first and second elongate members, each with a finned end plate, and an outer molded spacer (not shown).

FIG. 11 is an enlarged front elevational view of the assembly of FIG. 10 with the outer molded spacer shown in phantom.

FIG. 12 is an enlarged front elevational view, similar to FIG. 11, with portions broken away to show the detail thereof.

### DETAILED DESCRIPTION OF THE INVENTION

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure. It is also noted that any reference to the words top, bottom, up and down, and the like, in this application refers to the alignment shown in the various drawings, as well as the normal connotations applied to such devices, and is not intended to restrict positioning of the connecting member assemblies of the application and cooperating bone anchors in actual use.

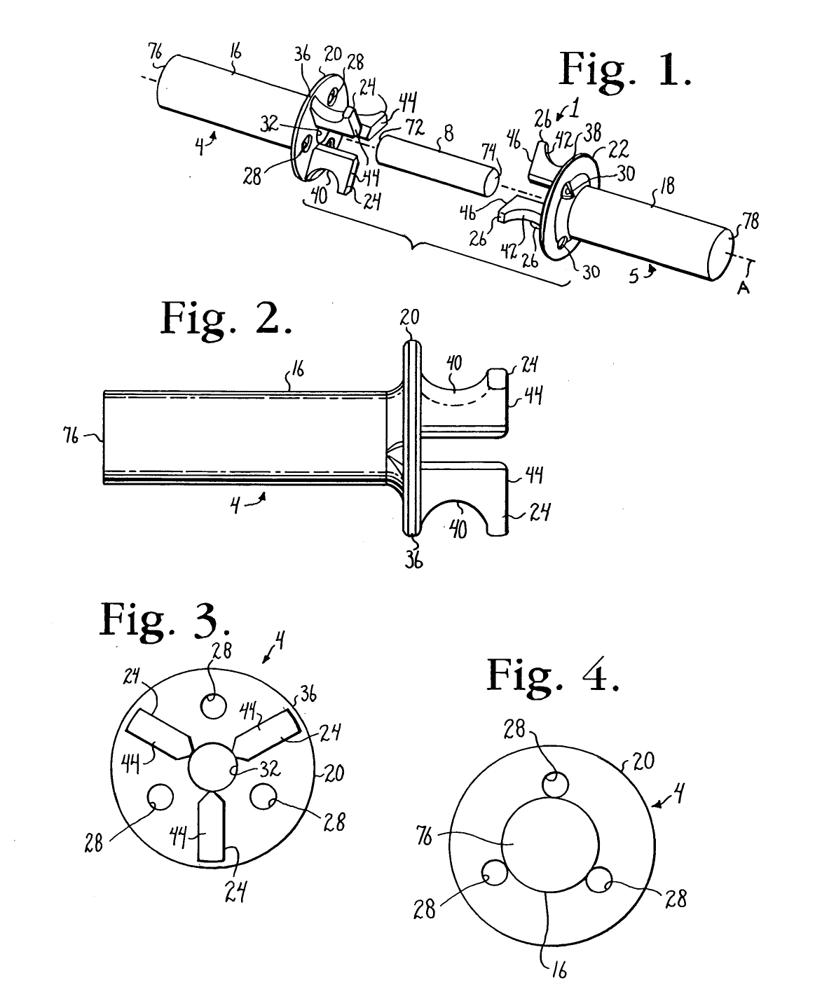

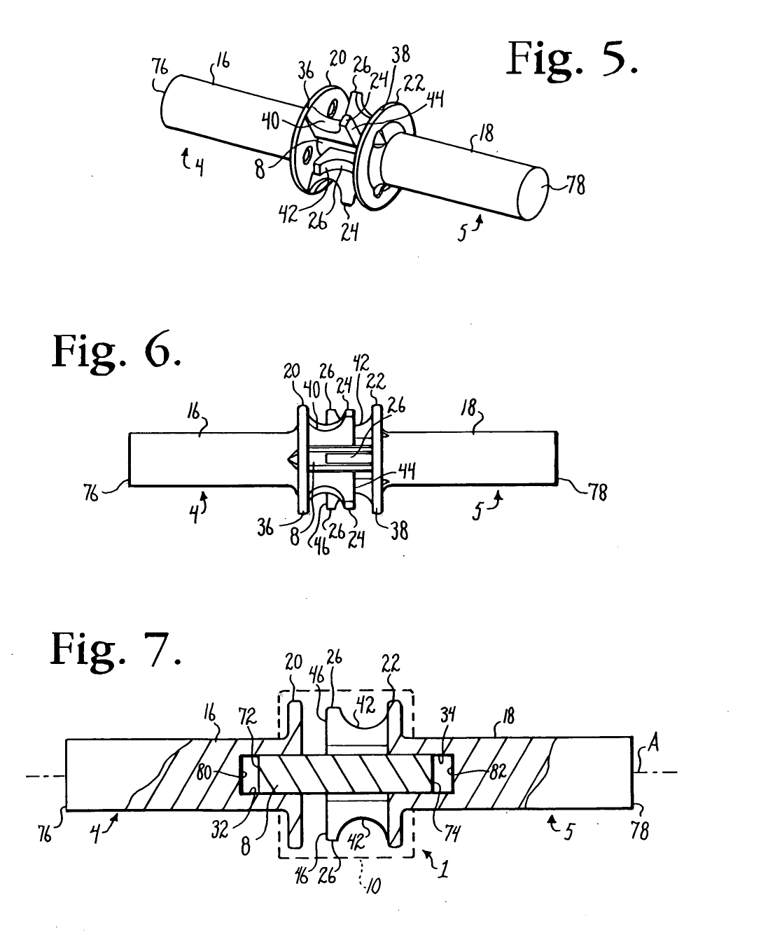

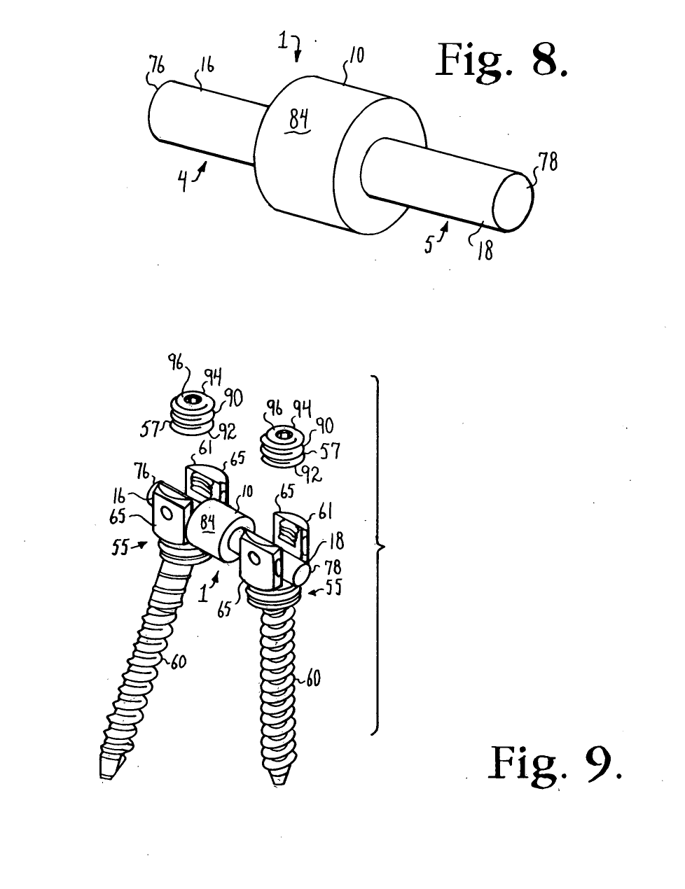

With reference to FIGS. 1-9, the reference numeral 1 generally designates a non-fusion dynamic stabilization longitudinal connecting member assembly according to the present invention. The connecting member assembly 1 includes first and second substantially identical elongate segments, generally 4 and 5, an optional inner core or floating pin segment 8, and an outer sleeve or spacer 10. The illustrated inner pin 8 is cylindrical and substantially solid, having a central longitudinal axis A that is also the central longitudinal axis A of the entire assembly 1 when the spacer 10 is molded thereon, connecting the segments 4 and 5 and the pin 8. The pin 8 provides stability to the assembly 1, particularly with respect to torsional and shear stresses placed thereon. It is noted that the pin 8 may be omitted or replaced by one or more cords, cables or other elongate members of a variety of cross-sectional shapes, including, but not limited to oval, rectangular, square and other polygonal and curved shapes. Such cords or cables may be attached to one of the segments 4 or 5 and tensioned prior to molding of the spacer 10.

With particular reference to FIGS. 1-4 the elongate segments 4 and 5 further include respective bone attachment end portions 16 and 18, respective end plates 20 and 22 having respective integral hooked fin or wing members 24 and 26. In the illustrated embodiment, there are three equally spaced fins 24 and 26 extending generally along the axis A from the respective plates 20 and 22. However, in other embodiments according to the invention there may be more than three or less than three hooked fins 24 and 26. Each plate 20 and 22 also includes three apertures or through bores 28 and 30, respectively, spaced substantially equally between the respective fins 24 and 26. The through bores 28 and 30 extend substantially parallel to the axis A. The segments 4 and 5 further include a respective central aperture 32 and 34, formed in the respective plates 20 and 22 and extending into the respective end portion 16 and 18. The apertures 32 and 34 are operatively located along the axis A and are sized and shaped to slidingly receive the inner core or pin 8 as best shown in FIG. 7.

As best shown in FIG. 3, each of the hooked fins 24, as well as the hooked fins 26, extend axially away from the respective plate 20, 22 (along the axis A) and also extend radially from the respective central aperture 32, 34 to or substantially near a respective outer peripheral substantially cylindrical surface 36 and 38 of the respective plates 20 and 22. Near the peripheral surfaces 36 and 38, the respective fins 24 and 26 include a curved concave or C-shaped hooked surface 40 and 42, respectively, such surface facing outwardly away from the axis A and running from the respective plates 20 and 22 to near respective end surfaces 44 and 46. When the segments 4 and 5 are assembled and set in place by the molded spacer 10, the surfaces 44 are near and in substantially uniform spaced relation with the plate 22 and the surfaces 46 are near and in substantially uniform spaced relation with the plate 20. The hooked surfaces 40 and 42 provide structure for mechanical cooperation and attachment with the molded spacer 10 as will be discussed in greater detail below. Also, as will be described in greater detail below, the spacer 10 is molded about the hooked fins 24 and 26, about the pin 8 and through the apertures or bores 28 and 30 of the respective plates 20 and 22 in a manner so as to result in a mechanically connected structure, the elastomeric material completely surrounding the plates 20 and 22 as well as the fins 24 and 26. In certain embodiments, the elastomeric material of the molded spacer 10 may be adhered to the fin, pin and plate surfaces and not completely surround the plants 20 and 22. An adhesive may also be added to provide such adherence between the spacer 10 and the plates and fins. Alternatively, in certain embodiments a coating or sleeve may be placed around the pin 8 prior to molding so that the pin 8 is spaced from the spacer 10 and thus slidably movable with respect to the spacer 10.

The dynamic connecting member assembly 1 cooperates with at least a pair of bone anchors, such as the polyaxial bone screws, generally 55 and cooperating closure structures 57 shown in FIG. 9, the assembly 1 being captured and fixed in place at the end portions 16 and 18 by cooperation between the bone screws 55 and the closure structures 57 with the spacer 10 being disposed between the bone screws 55.

Because the illustrated end portions 16 and 18 are substantially solid and cylindrical, the connecting member assembly 1 may be used with a wide variety of bone anchors already available for cooperation with rigid rods including fixed, monoaxial bone screws, hinged bone screws, polyaxial bone screws, and bone hooks and the like, with or without compression inserts, that may in turn cooperate with a variety of closure structures having threads, flanges, or other structure for fixing the closure structure to the bone anchor, and may include other features, for example, break-off tops and inner set screws. It is foreseen that the portions 16 and 18 may in other embodiments of the invention have other cross-sectional shapes, including, but not limited to oval, square, rectangular and other curved or polygonal shapes. The bone anchors, closure structures and the connecting member assembly 1 are then operably incorporated in an overall spinal implant system for correcting degenerative conditions, deformities, injuries, or defects to the spinal column of a patient.

The illustrated polyaxial bone screws 55 each include a shank 60 for insertion into a vertebra (not shown), the shank 60 being pivotally attached to an open receiver or head 61. The shank 60 includes a threaded outer surface and may further include a central cannula or through-bore disposed along an axis of rotation of the shank to provide a passage through the shank interior for a length of wire or pin inserted into the vertebra prior to the insertion of the shank 60, the wire or pin providing a guide for insertion of the shank 60 into the vertebra. The receiver 61 has a pair of spaced and generally parallel arms 65 that form an open generally U-shaped channel therebetween that is open at distal ends of the arms 65. The arms 65 each include radially inward or interior surfaces that have a discontinuous guide and advancement structure mateable with cooperating structure on the closure structure 57. The guide and advancement structure may take a variety of forms including a partial helically wound flangeform, a buttress thread, a square thread, a reverse angle thread or other thread like or non-thread like helically wound advancement structure for operably guiding under rotation and advancing the closure structure 57 downward between the receiver arms 65 and having such a nature as to resist splaying of the arms 65 when the closure 57 is advanced into the U-shaped channel. For example, a flange form on the illustrated closure 57 and cooperating structure on the arms 65 is disclosed in Applicant's U.S. Pat. No. 6,726,689, which is incorporated herein by reference.

The shank 60 and the receiver 61 may be attached in a variety of ways. For example, a spline capture connection as described in U.S. Pat. No. 6,716,214, and incorporated by reference herein, is used for the embodiment disclosed herein. Polyaxial bone screws with other types of capture connections may also be used according to the invention, including but not limited to, threaded connections, frictional connections utilizing frusto-conical or polyhedral capture structures, integral top or downloadable shanks, and the like. Also, as indicated above, polyaxial and other bone screws for use with connecting members of the invention may have bone screw shanks that attach directly to the segments 16 and 18 may include compression members or inserts that cooperate with the bone screw shank, receiver and closure structure to secure the connecting member assembly to the bone screw and/or fix the bone screw shank at a desired angle with respect to the bone screw receiver that holds the longitudinal connecting member assembly. Furthermore, although the closure structure 57 of the present invention is illustrated with the polyaxial bone screw 55 having an open receiver or head 61, it foreseen that a variety of closure structure may be used in conjunction with any type of medical implant having an open or closed head, including monoaxial bone screws, hinged bone screws, hooks and the like used in spinal surgery.

To provide a biologically active interface with the bone, the threaded shank 60 may be coated, perforated, made porous or otherwise treated. The treatment may include, but is not limited to a plasma spray coating or other type of coating of a metal or, for example, a calcium phosphate; or a roughening, perforation or indentation in the shank surface, such as by sputtering, sand blasting or acid etching, that allows for bony ingrowth or ongrowth. Certain metal coatings act as a scaffold for bone ingrowth. Bio-ceramic calcium phosphate coatings include, but are not limited to: alpha-tri-calcium phosphate and beta-tri-calcium phosphate (Ca₃ (PO₄)₂, tetra-calcium phosphate (Ca₄P₂O₉), amorphous calcium phosphate and hydroxyapatite (Ca₁₀(PO₄)₆(OH)₂). Coating with hydroxyapatite, for example, is desirable as hydroxyapatite is chemically similar to bone with respect to mineral content and has been identified as being bioactive and thus not only supportive of bone ingrowth, but actively taking part in bone bonding.

The longitudinal connecting member assembly 1 illustrated in FIGS. 1-9 is elongate, with the attachment portion 16, the plate 20 and the fins 24 being integral and the attachment portion 18, the plate 22 and the fins 26 being integral. The inner pin 8 is slidingly received in both the portion 16 and the portion 18. The segments 4 and 5 and the core 8 are preferably made from metal, metal alloys or other suitable materials, including plastic polymers such as polyetheretherketone (PEEK), ultra-high-molecular weight-polyethylene (UHMWP), polyurethanes and composites. Furthermore, in embodiments wherein the segments 4 and 5 are made from a plastic, such as PEEK, the pin 8 may advantageously be made from a material, such as tantalum, to provide an x-ray marker. The spacer 10 may be made of a variety of materials including plastics and composites. The illustrated spacer 10 is a molded thermoplastic elastomer, for example, polyurethane or a polyurethane blend; however, any suitable polymer material may be used.

Specifically, in the illustrated embodiment, the pin 8 and the end portions 16 and 18 are all substantially solid, smooth and uniform cylinders or rods, each of a uniform circular cross-section. It is foreseen that in some embodiments, the pin 8 and the segments 4 and 5 may include a small central lumen along an entire length thereof and opening at each end thereof to allow for threading therethrough and subsequent percutaneous implantation of the member 1. The illustrated pin 8 has an end 72 and an opposite end 74, with the solid end portion 16 terminating at an end 76 and the solid end portion 18 terminating at an end 78. The portions 16 and 18 are each sized and shaped to be received in the channel formed between the arms 65 of a bone screw 55 with the plates 20 and 22 and the molded spacer 10 disposed between cooperating bone screws 55.

As shown in FIG. 7, the pin 8 ends 72 and 74 are spaced from end surfaces 80 and 82 defining respective central apertures 32 and 34. It is foreseen that alternatively, an elastomeric cushion may be inserted between the pin end 72 and the surface 80 and the pin end 74 and the surface 82, thus functioning as a damper to axially directed compressive forces placed on the assembly 1.

The spacer 10 advantageously cooperates with the plates 20 and 22, the fins 24 and 26 and the pin 8 to provide a flexible or dynamic segment that allows for bending, torsion, compression and distraction of the assembly 1. The spacer 10 further provides a smooth substantially cylindrical surface that protects a patient's body tissue from damage that might otherwise occur with, for example, a spring-like dynamic member.

In the embodiment shown, the molded spacer 10 is fabricated about the plates 20 and 22 and the fins 24 and 26, as will be described more fully below, and in the presence of the pin 8, with molded plastic flowing about the plates, pin and fins. The formed elastomer is substantially cylindrical in outer form with an external substantially cylindrical surface 84 that has the same or substantially similar diameter as the diameter of the outer cylindrical surfaces 36 and 38 of the respective stop plates 20 and 22. It is foreseen that in some embodiments, the spacer may be molded to be of square, rectangular or other outer and inner cross-sections including curved or polygonal shapes. The spacer 10 may further include one or more compression grooves (not shown) formed in the surface 84. During the molding process a sleeve or other material (not shown) may be placed about the pin 8 so that the spacer 10 has in internal surface of a slightly greater diameter than an outer diameter of the pin 8, allowing for axially directed sliding movement of the spacer 10 with respect to the pin 8.

As stated above, it is foreseen that in other embodiments of the invention, the pin 8 may be omitted, resulting in a more flexible assembly 1. The pin 8 may be replaced with tensioned or un-tensioned cords or cables that are affixed to one or both of the segments 4 and 5. The pin 8 may be made from an elastomer. The pin 8 may be fixed to one of the segments 4 or 5 and/or extend through the other segment, providing an elongate inner core extending along a substantial length of the assembly, that may be pre-tensioned, if desired. In such embodiments, elastomeric end bumpers may be added to the assembly. The fins 24 and 26 may also be modified. For example, fewer, thicker fins may be utilized or a greater number of thinner fins may be used. Fewer fins may desirably allow for more torsional play in the assembly 1, whereas a greater number of fins may result in a tighter, less flexible assembly with the fins abutting one another when under fairly small torsional loads. In other embodiments, the fins may be solid and not include the c-shaped surface, allowing for more flexibility in distraction and compression. The fins may also have central opening or fenestrations.

With reference to FIG. 9, the closure structure 57 can be any of a variety of different types of closure structures for use in conjunction with the present invention with suitable mating structure on the interior surface of the upstanding arms 65 of the receiver 61. The illustrated closure structure 57 is rotatable between the spaced arms 65, but could be a twist-in or a slide-in closure structure. As described above, the illustrated closure structure 57 is substantially cylindrical and includes an outer helically wound guide and advancement structure in the form of a flange form 90 that operably joins with the guide and advancement structure disposed on the interior of the arms 65. The illustrated closure structure 57 includes a lower or bottom surface 92 that is substantially planar and may include a point and/or a rim protruding therefrom for engaging the section 16 or 18 outer cylindrical surface. The closure structure 57 has a top surface 94 with an internal drive feature 96, that may be, for example, a star-shaped drive aperture sold under the trademark TORX. A driving tool (not shown) sized and shaped for engagement with the internal drive feature 96 is used for both rotatable engagement and, if needed, disengagement of the closure 57 from the arms 65. The tool engagement structure 96 may take a variety of forms and may include, but is not limited to, a hex shape or other features or apertures, such as slotted, tri-wing, spanner, two or more apertures of various shapes, and the like. It is also foreseen that the closure structure 57 may alternatively include a break-off head designed to allow such a head to break from a base of the closure at a preselected torque, for example, 70 to 140 inch pounds. Such a closure structure would also include a base having an internal drive to be used for closure removal.

In use, at least two bone screws 55 are implanted into vertebrae for use with the longitudinal connecting member assembly 1. Each vertebra may be pre-drilled to minimize stressing the bone. Furthermore, when a cannulated bone screw shank is utilized, each vertebra will have a guide wire or pin (not shown) inserted therein that is shaped for the bone screw cannula of the bone screw shank 60 and provides a guide for the placement and angle of the shank 60 with respect to the cooperating vertebra. A further tap hole may be made and the shank 60 is then driven into the vertebra by rotation of a driving tool (not shown) that engages a driving feature at or near a top of the shank 60. It is foreseen that the screws 55 and the longitudinal connecting member 1 can be inserted in a percutaneous or minimally invasive surgical manner.

With particular reference to FIGS. 1-8, the longitudinal connecting member assembly 1 is assembled by inserting the end 72 of the pin 8 within the aperture 32 of the segment 4 and the end 74 of the pin within the aperture 34 of the segment 5. The fins 24 and 26 are manipulated to be evenly spaced with a desired uniform substantially equal space between the fin ends 46 and the plate 20 and the fin ends 44 and the plate 22. This is performed in a factory setting with the end portions 16 and 18 held in a jig or other holding mechanism that frictionally engages and holds the sections 16 and 18, for example, and the spacer 10 is molded about the plates 20 and 22 as well as the fins 24 and 26 as shown in phantom in FIG. 7. The elastomer of the spacer 10 flows through the plate through bores 28 and 30 as well as around and about each of the fins 24 and 26, the resulting molded spacer 10 surrounding all of the surfaces of the plates 20 and 22 as well as all of the surfaces of the fins 24 and 26. If desired, prior to molding, a sheath or coating may be placed about the pin 8 so that the spacer 10 material does not contact the pin 8. However, in other embodiments of the invention, the elastomer is allowed to flow about and contact the pin 8. The jig or holding mechanism is released from the portions 16 and 18 after the molding of the spacer 10 is completed.

With reference to FIG. 9, the assembly 1 is eventually positioned in an open or percutaneous manner in cooperation with the at least two bone screws 55 with the spacer 10 disposed between the two bone screws 55 and the end portions 16 and 18 each within the U-shaped channels of the two bone screws 55. A closure structure 57 is then inserted into and advanced between the arms 65 of each of the bone screws 55. The closure structure 57 is rotated, using a tool (not shown) engaged with the inner drive 96 until a selected pressure is reached at which point the portion 16 or 18 is urged toward, but not completely seated in the U-shaped channels of the bone screws 55. For example, about 80 to about 120 inch pounds pressure may be required for fixing the bone screw shank 60 with respect to the receiver 61 at a desired angle of articulation.

The assembly 1 is thus substantially dynamically loaded and oriented relative to the cooperating vertebra, providing relief (e.g., shock absorption) and protected movement with respect to flexion, extension, distraction, compressive, torsion and shear forces placed on the assembly 1 and the two connected bone screws 55. The spacer 10 and cooperating pin 8 and fins 24 and 26 allows the assembly 1 to twist or turn, providing some relief for torsional stresses. The spacer 10 in cooperation with the fins 24 and 26, however limits such torsional movement as well as bending movement, compression and distraction, providing spinal support. The pin 8 further provides protection against sheer stresses placed on the assembly 1.

If removal of the assembly 1 from any of the bone screw assemblies 55 is necessary, or if it is desired to release the assembly 1 at a particular location, disassembly is accomplished by using the driving tool (not shown) with a driving formation cooperating with the closure structure 57 internal drive 96 to rotate and remove the closure structure 57 from the receiver 61. Disassembly is then accomplished in reverse order to the procedure described previously herein for assembly.

Eventually, if the spine requires more rigid support, the connecting member assembly 1 according to the invention may be removed and replaced with another longitudinal connecting member, such as a solid rod, having the same diameter as the end portions 16 and 18, utilizing the same receivers 61 and the same or similar closure structures 57. Alternatively, if less support is eventually required, a less rigid, more flexible assembly, for example, an assembly 1 made without the pin 8 or from a more flexible material, or with fewer fins, but with end portions having the same diameter as the portions 16 and 18, may replace the assembly 1, also utilizing the same bone screws 55.

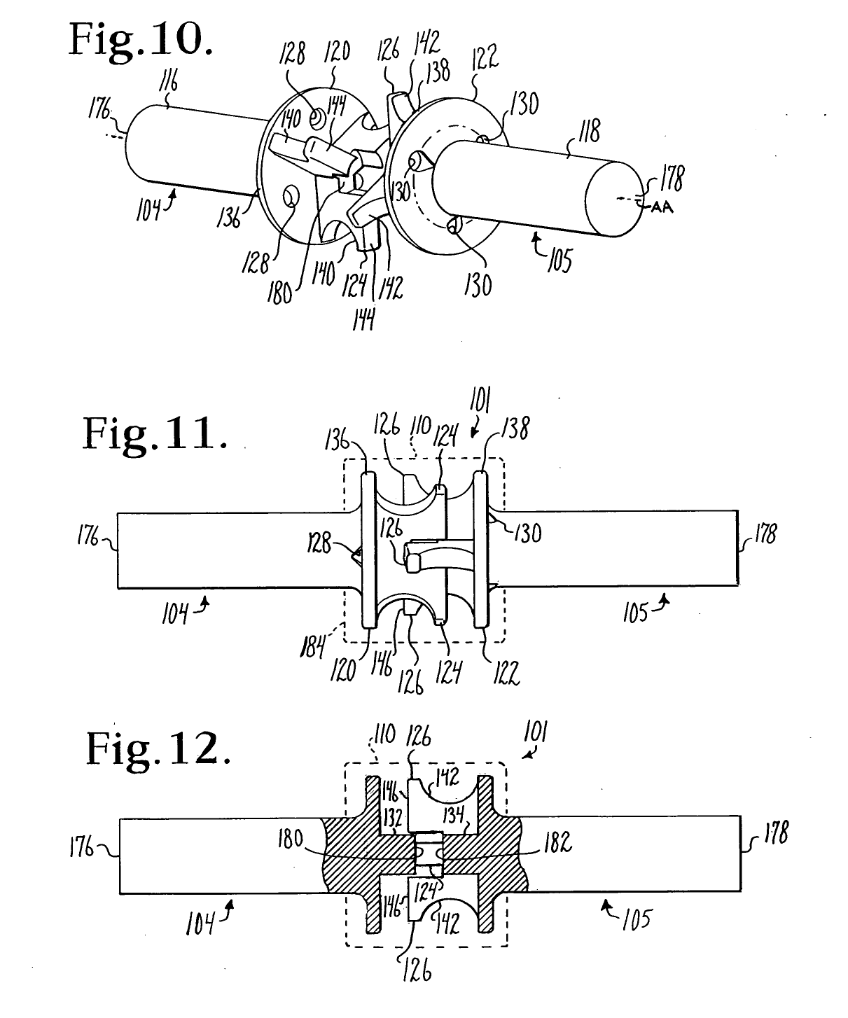

With reference to FIGS. 10-12, the reference numeral 101 generally designates an alternative embodiment of a non-fusion dynamic stabilization longitudinal connecting member assembly according to the present invention. The connecting member assembly 101 includes first and second substantially identical elongate segments, generally 104 and 105 and an outer over-molded sleeve or spacer 110, the segments 104 and 105 generally aligned along an axis AA. The assembly 101 is substantially similar to the assembly 1 with the exception that the assembly 101 does not include an inner floating pin or apertures for receiving such a pin. The elongate segments 104 and 105 include respective bone attachment end portions 116 and 118, respective end plates 120 and 122 having respective integral hooked fin or wing members 124 and 126. In the illustrated embodiment, there are three equally spaced fins 124 and 126 extending generally along the axis AA from the respective plates 120 and 122. However, in other embodiments according to the invention there may be more than three or less than three hooked fins 124 and 126. Each plate 120 and 122 also includes three apertures or through bores 128 and 130, respectively, spaced substantially equally between the respective fins 124 and 126. The through bores 128 and 130 extend substantially parallel to the axis AA. The segments 104 and 105 further include a respective central support member 132 and 134, integral with and extending axially away from the respective plates 120 and 122, the respective fins 124 and 126 extending radially from the respective end pieces 120 and 122. As best shown in FIGS. 10 and 11, each of the hooked fins 124, as well as the hooked fins 126, extend axially away from the respective plate 120, 122 (along the axis AA) and also extend radially from the respective central support member 132 and 134 to or substantially near a respective outer peripheral substantially cylindrical surface 136 and 138 of the respective plates 120 and 122. Near the peripheral surfaces 136 and 138, the respective fins 124 and 126 include a curved concave or C-shaped hooked surface 140 and 142, respectively, such surface facing outwardly away from the axis AA and running from the respective plates 120 and 122 to near respective end surfaces 144 and 146. When the segments 104 and 105 are assembled and set in place by the over-molded spacer 110, the surfaces 144 are near and in substantially uniform spaced relation with the plate 122 and the surfaces 146 are near and in substantially uniform spaced relation with the plate 120. The hooked surfaces 140 and 142 provide structure for mechanical cooperation and attachment with the molded spacer 110. Also, substantially similar or identical to the spacer 10 and fins 24 and 26 of the assembly 1, the spacer 110 is molded about the hooked fins 124 and 26 and through the apertures or bores 128 and 130 of the respective plates 120 and 122 in a manner so as to result in a mechanically connected structure, the elastomeric material at least partially and preferably completely surrounding the plates 120 and 122 as well as the fins 124 and 126 with the elastomer also filling the gap between and around the spaced central supports 132 and 134. In certain embodiments, the elastomeric material of the molded spacer 110 may be adhered to the fin and plate surfaces and not completely surround the plates 120 and 122. An adhesive may also be added to provide such adherence between the spacer 110 and the plates and fins.

The dynamic connecting member assembly 101 cooperates with at least a pair of bone anchors, such as the polyaxial bone screws, generally 55 and cooperating closure structures 57 shown in FIG. 9 and previously described herein with respect to the assembly 1. The portion 116 includes an end 176 that may be cut to any desired length. The portion 118 has an end 178 that may be cut to any desired length. It is foreseen that the portions 116 and 118 may in other embodiments of the invention have other cross-sectional shapes, including, but not limited to oval, square, rectangular and other curved or polygonal shapes. The bone anchors, closure structures and the connecting member assembly 101 are then operably incorporated in an overall spinal implant system for correcting degenerative conditions, deformities, injuries, or defects to the spinal column of a patient.

The spacer 110 advantageously cooperates with the plates 120 and 122 and the fins 124 and 126 to provide a flexible or dynamic segment that allows for bending, torsion, compression and distraction of the assembly 101. The spacer 110 further provides a smooth substantially cylindrical surface that protects a patient's body tissue from damage that might otherwise occur with, for example, a spring-like dynamic member. In the embodiment shown, the molded spacer 110 is fabricated about the plates 120 and 122, the fins 124 and 126 and between respective end surfaces 180 and 182 of central supports 132 and 134. The formed elastomer is substantially cylindrical in outer form with an external substantially cylindrical surface 184 that has the same or slightly larger diameter as the diameter of the outer cylindrical surfaces 136 and 138 of the respective stop plates 120 and 122. It is foreseen that in some embodiments, the spacer may be molded to be of square, rectangular or other outer and inner cross-sections including curved or polygonal shapes. The spacer 110 may further include one or more compression grooves (not shown) formed in the surface 184.

In such embodiments, elastomeric end bumpers may be added to the assembly. The fins 124 and 126 may also be modified. For example, fewer, thicker fins may be utilized or a greater number of thinner fins may be used. Fewer fins may desirably allow for more torsional play in the assembly 101, whereas a greater number of fins may result in a tighter, less flexible assembly with the fins abutting one another when under fairly small torsional loads. In other embodiments, the fins may be solid and not include the c-shaped surface, allowing for more flexibility in distraction and compression. The fins may also have central opening or fenestrations.

The longitudinal connecting member assembly 101 is assembled by facing the end surfaces 180 and 182 towards one another and moving the fins 124 and 126 into slightly overlapping position with respect to the axis AA and in evenly spaced radial relation. This is performed in a factory setting with the end portions 116 and 118 held in a jig or other holding mechanism that frictionally engages and holds the sections 116 and 118, for example, and the spacer 110 is molded about the plates 120 and 122 as well as the fins 124 and 126 as shown in phantom in FIGS. 11 and 12. The elastomer of the spacer 110 flows through the plate through bores 128 and 130 as well as around and about each of the fins 124 and 126, the resulting molded spacer 110 surrounding all of the surfaces of the plates 120 and 122 as well as all of the surfaces of the fins 124 and 126. The jig or holding mechanism is released from the portions 116 and 118 after the molding of the spacer 110 is completed.

The assembly 101 is eventually positioned in an open or percutaneous manner in cooperation with the at least two bone screws 55 with the spacer 110 disposed between the two bone screws 55 and the end portions 116 and 118 each within the U-shaped channels of the two bone screws 55. A closure structure 57 is then inserted into and advanced between the arms 65 of each of the bone screws 55. The closure structure 57 is rotated, using a tool (not shown) engaged with the inner drive 96 until a selected pressure is reached at which point the portion 116 or 118 is urged toward, but not completely seated in the U-shaped channels of the bone screws 55. For example, about 80 to about 120 inch pounds pressure may be required for fixing the bone screw shank 60 with respect to the receiver 61 at a desired angle of articulation.

The assembly 101 is thus substantially dynamically loaded and oriented relative to the cooperating vertebra, providing relief (e.g., shock absorption) and protected movement with respect to flexion, extension, distraction, compressive, torsion and shear forces placed on the assembly 101 and the two connected bone screws 55. The spacer 110 in cooperation with the fins 124 and 126 limits torsional movement as well as bending movement, compression and distraction, providing spinal support.

If removal of the assembly 101 from any of the bone screw assemblies 55 is necessary, or if it is desired to release the assembly 101 at a particular location, disassembly is accomplished by using the driving tool (not shown) with a driving formation cooperating with the closure structure 57 internal drive 96 to rotate and remove the closure structure 57 from the receiver 61. Disassembly is then accomplished in reverse order to the procedure described previously herein for assembly.

It is to be understood that while certain forms of the present invention have been illustrated and described herein, it is not to be limited to the specific forms or arrangement of parts described and shown.

What is claimed and desired to be secured by Letters Patent is as follows:

1. In a medical implant assembly having at least two bone attachment structures cooperating with a longitudinal connecting member, the improvement wherein the longitudinal connecting member comprises:
a) first and second elongate segments, the segments aligned along a central axis, each segment having at least one fin extending axially therefrom and radially from the axis, the fins in spaced, overlapping relation along the axis; and
b) a molded elastomer substantially surrounding each fin.

2. The improvement of claim 1 wherein the at least one fin is a plurality of fins.

3. The improvement of claim 1 wherein the at least one fin is at least a pair of fins on each elongate segment, the fins of the first segment disposed between the fins of the second segment.

4. The improvement of claim 3 wherein the fins are in substantially equal spaced relation to one another.

5. The improvement of claim 1 wherein the at least one fin has a concave surface.

6. The improvement of claim 5 wherein the concave surface faces outwardly away from the axis.

7. The improvement of claim 1 further comprising an inner floating pin.

8. The improvement of claim 7 wherein the inner floating pin extends into apertures of the first and second segments.

9. The improvement of claim 1 wherein each elongate segment is cylindrical.

10. The improvement of claim 1 wherein each elongate segment has at least one end plate and the at least one fin extends axially from the end plate.

11. The improvement of claim 10 wherein the molded elastomer surrounds each end plate.

12. In a medical implant assembly having at least two bone anchors cooperating with a longitudinal connecting member, the improvement wherein the longitudinal connecting member comprises:
a) a first elongate member having a first axis, the member sized and shaped for attachment to at least one bone anchor, the elongate member having a first end plate and a first curvate fin fixed to the end plate, the curvate fin extending along the first axis and radially outward from the first axis;
b) a second elongate member having a second axis, the second member sized and shaped for attachment to at least one bone anchor, the second elongate member having a second end plate and a second curvate fin fixed to the second end plate, the second curvate fin extending along the second axis and radially outward from the second axis; and
c) a molded elastomer surrounding the first and second curvate fins and holding the fins in substantially spaced relation with one another, the fins in at least partial overlapping relation to one another along the first and second axes.

13. The improvement of claim 12 wherein the elastomer surrounds at least the first end plate.

14. The improvement of claim 12 wherein the elastomer surrounds the first and second end plates.

15. The improvement of claim 12 wherein the first fin is a plurality of fins and the second fin is a plurality of fins, each first fin being at least partially disposed between a pair of second fins.

16. The improvement of claim 12 wherein a first aperture is formed in the first plate and a portion of the first member and a second aperture is formed in the second plate and a portion of the second member and an elongate pin is slidingly disposed in the first and second apertures.

17. The improvement of claim 12 wherein each end plate has at least one aperture and the elastomer is disposed in the at least one aperture.

18. In a medical implant assembly having at least two bone anchors cooperating with a longitudinal connecting member, the improvement wherein the longitudinal connecting member comprises:
a) a first elongate member having a first axis, the member sized and shaped for attachment to at least one bone anchor, the elongate member having at least three curvate fins extending along a first axis and radially outwardly from the first axis;
b) a second elongate member having a second axis, the second member sized and shaped for attachment to at least one bone anchor, the second elongate member having at least three curvate fins extending along the second axis and radially outwardly from the first axis, the fins of the first member being at least partially disposed between the fins of the second member; and
c) a molded elastomer disposed about and between all of the curvate fins.
